(19) United States
(12) Patent Application Publication
(10) Pub. No.: US 2007/0015838 A1
(43) Pub. Date: Jan. 18, 2007

(54) Cyclopentane n-lower alkyl heptenamide-5-cis-2-(3alpha-hydroxy-5-phenylpentyl)-3, 5-dihydroxy, [1alpha, 2beta, 3alpha, 5alpha] compounds as agents for lowering intraocular pressure

(75) Inventor: Martin A. Voet, San Juan Capistrano, CA (US)

Correspondence Address: Robert J. Baran (T2-7H);ALLERGAN, INC., Legal Department, 2525 Dupont Drive, Irvine, CA, 92612, US

(73) Assignee: Allergan, Inc.

(21) Appl. No.: 11/455,583

(22) Filed: Jul. 10, 2006

Related U.S. Application Data
(60) Provisional application No. 60/699,813, filed on Jul. 14, 2005.

(51) Int. Cl. A61K 31/165; A61K 31/557

(52) U.S. Cl. 514/622

(57) ABSTRACT

The present invention provides a method of treating ocular hypertension or glaucoma which comprises administering to an animal having ocular hypertension or glaucoma therapeutically effective amount of a compound represented by the general formula I; 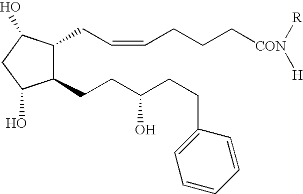
wherein R is a lower alkyl radical, i.e. a C₁-C₆ alkyl radical, e.g. a methyl, ethyl or propyl radical. Most preferably said compound is (Z)-7-[1R, 2R, 2R, 5S)-3,5-Dihydroxy-2-[1E, 3S)-3-hydroxy-5-phenyl-1-pentyl]cyclopentyl]-5-N-ethylheptanamide.

### CROSS REFERENCE TO RELATED APPLICATIONS

This application is based on, and claims the benefit of, U.S. Provisional Application No. 60/699,813, filed Jul. 14, 2005, and which is incorporated herein by reference.

### FIELD OF THE INVENTION

The present invention relates to cyclopentane n-lower alkyl heptenamide-5-cis-2-(3a-hydroxy-5-phenylpentyl)-3,5-dihydroxy, [1α, 2β, 3α, 5α] compounds as potent ocular hypotensives that are particularly suited for the management of glaucoma.

### BACKGROUND OF THE INVENTION

### DESCRIPTION OF RELATED ART

Ocular hypotensive agents are useful in the treatment of a number of various ocular hypertensive conditions, such as post-surgical and post-laser trabeculectomy ocular hypertensive episodes, glaucoma, and as presurgical adjuncts.

Glaucoma is a disease of the eye characterized by increased intraocular pressure. On the basis of its etiology, glaucoma has been classified as primary or secondary. For example, primary glaucoma in adults (congenital glaucoma) may be either open-angle or acute or chronic angle-closure. Secondary glaucoma results from pre-existing ocular diseases such as uveitis, intraocular tumor or an enlarged cataract.

The underlying causes of primary glaucoma are not yet known. The increased intraocular tension is due to the obstruction of aqueous humor outflow. In chronic open-angle glaucoma, the anterior chamber and its anatomic structures appear normal, but drainage of the aqueous humor is impeded. In acute or chronic angle-closure glaucoma, the anterior chamber is shallow, the filtration angle is narrowed, and the iris may obstruct the trabecular meshwork at the entrance of the canal of Schlemm. Dilation of the pupil may push the root of the iris forward against the angle, and may produce pupilary block and thus precipitate an acute attack. Eyes with narrow anterior chamber angles are predisposed to acute angle-closure glaucoma attacks of various degrees of severity.

Secondary glaucoma is caused by any interference with the flow of aqueous humor from the posterior chamber into the anterior chamber and subsequently, into the canal of Schlemm. Inflammatory disease of the anterior segment may prevent aqueous escape by causing complete posterior synechia in iris bombe, and may plug the drainage channel with exudates. Other common causes are intraocular tumors, enlarged cataracts, central retinal vein occlusion, trauma to the eye, operative procedures and intraocular hemorrhage.

Considering all types together, glaucoma occurs in about 2% of all persons over the age of 40 and may be asymptotic for years before progressing to rapid loss of vision.

Certain eicosanoids and their derivatives have been reported to possess ocular hypotensive activity, and have been recommended for use in glaucoma management. Eicosanoids and derivatives include numerous biologically important compounds such as prostaglandins and their derivatives. Prostaglandins can be described as derivatives of prostanoic acid which have the following structural formula:

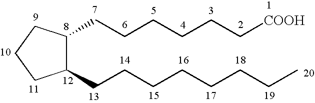

Various prostaglandin derivatives, e.g. latanoprost, travoprost, unoprostone isopropyl, etc. have been commercialized for lowering intraocular pressure and managing glaucoma. Recently, a prostamide, i.e. bimatoprost, has been marketed for treating increased eye pressure caused by open-angle glaucoma or ocular hypertension. Prostamides are structurally similar to prostaglandins but are biologically different. Prostamides, unlike prostaglandins, do not lower intraocular pressure by interaction with the prostaglandin receptor. (See U.S. Pat. No. 5,352,708, which hereby is incorporated by reference in its entirety.)

While prostaglandins and prostamides are effective in lowering intraocular pressure without significant intraocular side effects, ocular surface (conjunctival) hyperemia and foreign-body sensation have been associated with the topical ocular use of such compounds, in particular PGF_{2α} and its prodrugs, e.g., its 1-isopropyl ester, in humans.

Thus, it would be desirable to discover a prostamide or prostaglandin compound which effectively lowers intraocular pressure without causing hyperernia.

### BRIEF DESCRIPTION OF THE DRAWINGS FIGURES

FIG. 1 is a graph showing the effect of cyclopentane ethyl heptenamide-5-cis-2-(3α-hydroxy-5-phenylpentyl)-3,5-dihydroxy, [1α, 2β, 3α, 5α] and the corresponding 13, 14 unsaturated compound (open-square) vehicle as agents for lowering intraocular pressure (.circle-solid.) on dog intraocular pressure. Contralateral eyes received as a control. Points are mean values.+-.SEM; n=8.

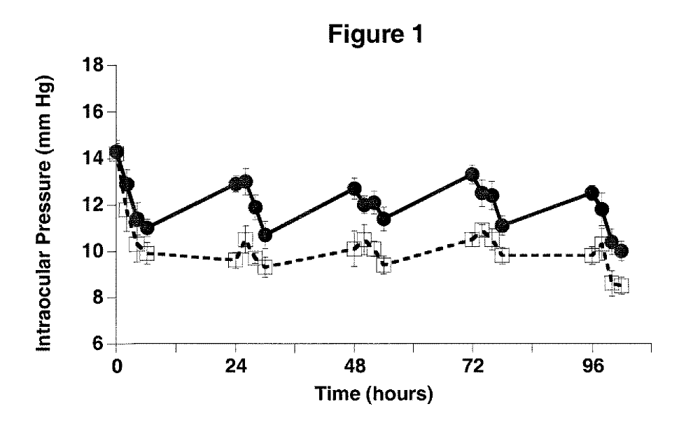

FIG. 2 is a graph showing the effect of cyclopentane ethyl heptenamide-5-cis-2-(3α-hydroxy-5-phenylpentyl)-3,5-dihydroxy, [1α, 2β, 3α, 5α] and corresponding 13, 14 unsaturated compound (open-square) vehicle as agents for lowering intraocular pressure (.circle-solid.) on dog pupil diameter. Contralateral eyes received the as a control. Points are mean values.+-.SEM; n=8.

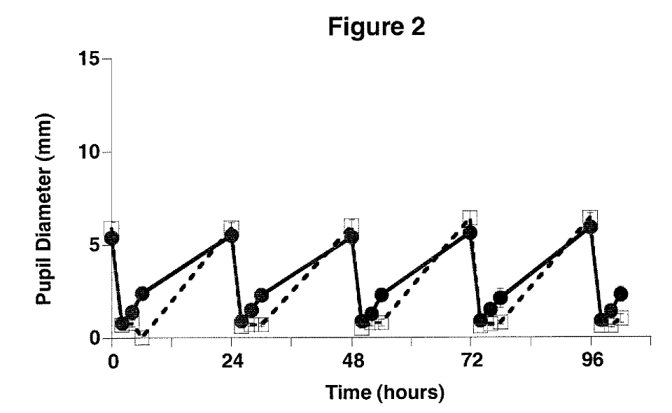

FIG. 3 is a graph showing the effect of cyclopentane ethyl heptenamide-5-cis-2-(3α-hydroxy-5-phenylpentyl)-3,5-dihydroxy, [1α, 2β, 3α, 5α] and the corresponding 13, 14 unsaturated compound (open-square) vehicle as agents for lowering intraocular pressure (.circle-solid.) on dog ocular surface hyperemia. Contralateral eyes received as a control. Points are mean values.+-.SEM; n=8.

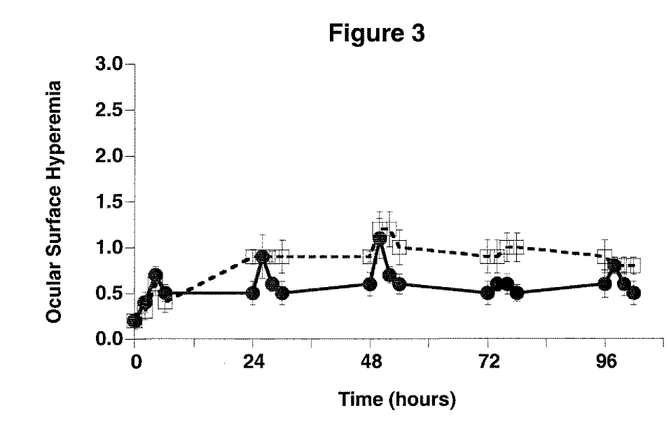

### SUMMARY OF THE INVENTION

The present invention concerns a method of treating ocular hypertension which comprises administering to a mammal having ocular hypertension a therapeutically effective amount of a compound of formula I 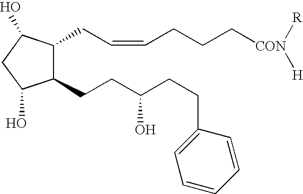
wherein R is a lower alkyl radical, i.e. a C₁-C₆ alkyl radical, e.g. a methyl, ethyl or propyl radical. Most preferably R is ethyl and the compound is (Z)-7-[1R,2R,2R,5S)-3,5-Dihydroxy-2-[1E,3S)-3-hydroxy-5-phenyl- 1 -pentyl]cyclopentyl]-5-N-ethylheptanamide. These compounds effectively lower intraocular pressure while having insignificant hyperemia, especially as compared to the corresponding 13, 14 unsaturated compound.

In another aspect of the invention, a ophthalmic solution comprising one or more of the above compounds of formula I in combination with an ophthalmically-acceptable vehicle is contemplated.

In a still further aspect, the present invention relates to a pharmaceutical product, comprising

a container adapted to dispense its contents in a metered form; and

an ophthalmic solution therein, as hereinabove defined.

Finally, certain of the compounds represented by the above formula, disclosed below and utilized in the method of the present invention are novel and unobvious.

### DETAILED DESCRIPTION OF THE INVENTION

The present invention relates to the use of cyclopentane n-lower alkyl heptenamide-5-cis-2-(3α-hydroxy-5-phenylpentyl)-3,5-dihydroxy, [1α, 2β, 3α, 5α] compounds as ocular hypotensives. The compounds used in accordance with the present invention are encompassed by the following structural formula I: 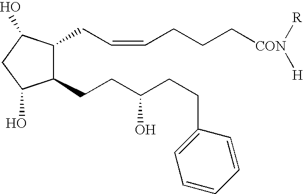
wherein R is a lower alkyl radical, i.e. a C₁-C₆ alkyl radical, e.g. a methyl, ethyl or propyl radical. Most preferably said compound is (Z)-7-[1R,2R,2R,5S)-3,5-Dihydroxy-2-[1E,3S)-3-hydroxy-5-phenyl-1-pentyl]cyclopentyl]-5-N-ethylheptanamide.

The above compounds of the present invention may be prepared by methods that are known in the art or according to the examples below.

Ophthalmic solutions may be prepared by combining a therapeutically effective amount of at least one compound according to the present invention, or a pharmaceutically acceptable acid addition salt thereof, as an active ingredient, with conventional ophthalmically acceptable pharmaceutical excipients, and by preparation of unit dosage forms suitable for topical ocular use. The therapeutically efficient amount typically is between about 0.0001 and about 5% (w/v), preferably about 0.001 to about 1.0% (w/v) in liquid formulations.

For ophthalmic application, preferably solutions are prepared using a physiological saline solution as a major vehicle. The pH of such ophthalmic solutions should preferably be maintained between 6.5 and 7.2 with an appropriate buffer system. The formulations may also contain conventional, pharmaceutically acceptable preservatives, stabilizers and surfactants.

Preferred preservatives that may be used in the ophthalmic solutions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 1-10 |
| buffer | 0.01-10 |
| pH adjustor | q.s. pH 4.5-7.5 |
| antioxidant | as needed |
| surfactant | as needed |
| purified water | as needed to make 100% |

The actual dose of the active compounds of the present invention depends on the specific compound; the selection of the appropriate dose is well within the knowledge of the skilled artisan.

The ophthalmic formulations of the present invention are conveniently packaged in forms suitable for metered application, such as in containers equipped with a dropper, to facilitate the application to the eye. Containers suitable for dropwise application are usually made of suitable inert, non-toxic plastic material, and generally contain between about 0.5 and about 15 ml solution.

### EXAMPLE 1

### (Z)-7-[1R,2R,2R,5S)-3,5-Dihydroxy-2-[1E,3S)-3-hydroxy-5-phenyl-1-pentyl]cyclopentyl]-5-N-ethylheptanamide.

Cyclopentane isopropyl heptenoate-5-cis-2 (3-hydroxy-5-phenyl-1-pentanyl)-3,5dihydroxy, [1α, 2β, 3^{α}, 5^{α}] is reacted with ethylamine according to the method of Example 12 of U.S. Pat. No. 5,352,708 to yield the named compound.

### EXAMPLE 2

### Intraocular Pressure (IOP)

Intraocular pressure studies in dogs involved pneumatonometry performed in conscious, Beagle dogs of both sexes (10-15 kg). The animals remained conscious throughout the study and were gently restrained by hand. Drugs were administered topically to one eye as a 25 .mu.L volume drop, the other eye received 25 .mu.L vehicle (0.1% polysorbate 80:10 mM TRIS) as a control. 0.1% proparacaine was used for corneal anesthesia during tonometry. Intraocular pressure was determined just before drug administration and at 2, 4 and 6 hr thereafter on each day of the 5 day study. Drug was administered immediately after the first IOP reading.

### Pupil Diameter

Dog pupil diameter was measured using an optistick (a mm ruler which included half-circle references of standard widths (mm) for reference. Gently restraining the dog by hand, pupil diameter was determined by matching a half-circle to the pupil in normal room light. In dogs with very dark pupils a specialized penlight was used, but only very briefly to avoid pupil constriction. Pupil diameter was measured at the same time as IOP and hyperemia.

### Ocular Surface Hyperemia

Ocular surface hyperemia was visually assessed and scored according to a system typically used clinically.

| Hyperemia Score | Assigned Value |
|---|---|
| <1 trace | 0.5 |
| 1 mild | 1 |
| moderate | 2 |
| severe | 3 |

Ocular surface hyperemia was evaluated at the same time points as intraocular pressure measurement. It should be noted that untreated dog eyes frequently have a pink/red tone. Thus, values of trace or even mild are not necessarily out of the normal range.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

1. A method of treating ocular hypertension or glaucoma which comprises administering to an animal having ocular hypertension or glaucoma a therapeutically effective amount of a compound represented by the general formula I; 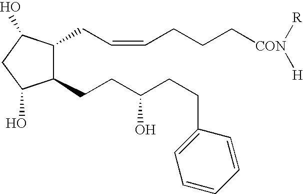
wherein R is a lower alkyl radical.

2. The method according to claim 1 wherein R is a C₁-C₆ alkyl radical.

3. The method according to claim 2 wherein R is selected from the group consisting of methyl, ethyl and propyl.

4. The method of claim 3 wherein said compound is (Z)-7-[1R,2R,2R,5S)-3,5-Dihydroxy-2-[1E,3S)-3-hydroxy-5-phenyl-1-pentyl]cyclopentyl]-5-N-ethylheptanamide.

5. The method of claim 1 wherein said animal is a human.

6. The method of claim 1 wherein said compound is administered as an aqueous solution.

7. The method of claim 6 wherein said aqueous solution comprises about 0.03% weight/volume of said compound.

8. The method of claim 6 wherein said solution is administered topically.

9. An ophthalmic solution comprising a therapeutically effective amount of a compound represented by the general Formula 1 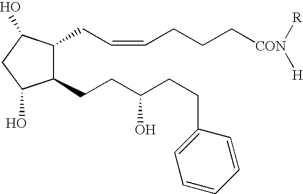
wherein R is a lower alkyl radical in combination with an ophthalmically acceptable pharmaceutical excipient.

10. The solution of claim 9 wherein R is a C₁-C₆ alkyl radical.

11. The solution of claim 10 wherein R is selected from the group consisting of methyl, ethyl and propyl.

12. The solution of claim 11 wherein said compound is (Z)-7-[1R,2R,2R,5S)-3,5-Dihydroxy-2-[1E,3S)-3-hydroxy-5-phenyl-1-pentyl]cyclopentyl]-5-N-ethylheptanamide.

13. The solution of claim 9 wherein said solution is an aqueous solution.

14. The solution of claim 13 wherein said compound comprises about 0.03% weight/volume of said solution.

15. A pharmaceutical product, comprising a container adapted to dispense the contents of said container in metered form; and an ophthalmic solution according to claim 5 in said container.

16. The product of claim 15 wherein R is a C₁-C₆ alkyl radical.

17. The product of claim 16 wherein R is selected from the group consisting of methyl, ethyl and propyl.

18. The product of claim 17 wherein said compound is (Z)-7-[1R,2R,2R,5S)-3,5-Dihydroxy-2-[1E,3S)-3-hydroxy-5-phenyl-1-pentyl]cyclopentyl]-5-N-ethylheptanamide.

19. The product of claim 15 wherein said solution is an aqueous solution.

20. The product of claim 19 wherein said compound comprises about 0.03% weight/volume of said solution.

21. A novel compound selected from the group consisting of compounds represented by formula I 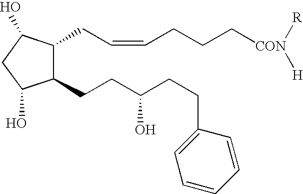
wherein R is a lower alkyl radical.

22. The compound of claim 21 wherein R is a C₁-C₆ alkyl radical.

23. The compound of claim 22 wherein R is selected from the group consisting of methyl, ethyl and propyl.

24. The compound of claim 23 wherein said compound is (Z)-7-[1R,2R,2R,5S)-3,5-Dihydroxy-2-[1E, 3S)-3-hydroxy-5-phenyl-1-pentyl]cyclopentyl]-5-N-ethylheptanamide.
